(19) United States
(12) Patent Application Publication
(10) Pub. No.: US 2026/0334788 A1
(43) Pub. Date: Oct. 29, 2026

(54) PHYSIOLOGICAL NAVIGATION IN MRI USING INHERENT SLAB-SELECTIVE FREE-INDUCTION-DECAY SIGNAL

(71) Applicant: The United States of America, as represented by the Secretary, Department of Health and Human Servic, Bethesda, MD (US)

(72) Inventors: Rajiv Ramasawmy, Bethesda, MD (US); Adrienne Elizabeth Campbell, Bethesda, MD (US); Ahsan Javed, Bethesda, MD (US); Daniel Herzka, Solon, OH (US)

(73) Assignee: The United States of America, as represented by the Secretary, Department of Health and Human Servic, Bethesda, MD (US)

(21) Appl. No.: 19/115,948

(22) PCT Filed: Sep. 28, 2023
(86) PCT No.: PCT/US2023/033997

§ 371 Date: Mar. 27, 2025

Related U.S. Application Data
(60) Provisional application No. 63/411,055, filed on Sep. 28, 2022.

(51) Int. Cl. A61B 5/00; A61B 5/08

(52) U.S. Cl. CPC A61B 5/0035; A61B 5/0816; A61B 5/725; A61B 5/7289

(57) ABSTRACT

A method and system for acquiring free-breathing magnetic resonance (MR) images are provided. The method utilizes a “free” navigator signal acquired by measuring MR signals during or following a slice select ramp down portion of an MRI pulse sequence. After processing the captured navigator signal with sampling-based and temporal filters, the resulting processed navigator signal may include information that correlates strongly with a respiratory cycle of a subject being imaged. Such navigator signal can be used retroactively to process the MRI image data to improve the image quality or resolve respiratory motion. This type of navigator signal can be used to allow fee-breathing of the subject during image acquisition, improving the comfort of the subject, while not significantly increasing the overall image capture time of many common MRI sequence.

### CROSS-REFERENCE TO RELATED APPLICATIONS

This application claims the benefit of U.S. Provisional Application No. 63/411,055, filed Sep. 28, 2022, which is herein incorporated by reference in its entirety.

### STATEMENT REGARDING FEDERALLY SPONSORED RESEARCH OR DEVELOPMENT

This invention was made with Government support under Grant Numbers Z01-HL006257 and Z01-HL006213 awarded by the National Institutes of Health. The Government has certain rights in this invention.

### BACKGROUND

In magnetic resonance imaging (MRI), physiological “navigation” is required for high-quality imaging which accounts for respiratory motion (and other motion), to avoid image artifacts and blurring. “Free-breathing” acquisitions are ideal for patient comfort and are more robust when patients are less compliant to holding their breath (i.e. children, or patients under anesthesia).

Recently, techniques for synchronizing image acquisition with timing of the patient's respiratory cycle using a navigator signal have been proposed. The navigation data can be acquired using the MRI system separate and distinct from capturing the image data. However, these techniques typically capture the navigation data in lieu of image data during normal acquisition timing cycles, which can increase the overall time to acquire the necessary image data. Typically, one or more MRI sequence cycles are used to acquire the navigator signal and, when the navigator signal indicates that no respiratory motion is present, the MRI sequence cycles capturing the image data of the structure under observation commence. Reducing the timing required to capture the necessary MRI image data can improve the comfort of a patient, who does not have to be confined in an MRI machine for as long. Thus, a technique for efficiently capturing a navigator signal during the same MRI sequence cycle as the image data is desired.

### SUMMARY

A navigator signal may be captured during a portion of the MRI pulse sequence after slice (or slab) selection and prior to k-space encoding for capturing image data using well-known MRI pulse sequence trajectories. The navigator signal enables a subject being imaged to continue breathing normally during image acquisition as motion compensation can be performed retroactively using post-processing techniques (e.g., binning) based on the captured navigator signal.

In accordance with a first aspect of the present disclosure, a method applied using a magnetic resonance imaging (MRI) apparatus is provided. The method includes: capturing data for a navigator signal between a slice select portion of an MRI sequence and a gradient refocus portion of the MRI sequence, processing the navigator signal to identify timing of a respiratory cycle of a subject being measured, and performing data reconstruction using the MRI apparatus based at least on the processed navigator signal. Performing data reconstruction can include performing retrospective MRI data binning based on the processed navigator signal. The slice select portion of the MRI sequence is a period where a current is passed through at least one gradient coil of the MRI apparatus while a radio frequency (RF) pulse is generated by a RF coil of the MRI apparatus.

In accordance with an embodiment of the first aspect, the data for the navigator signal is captured during a slice select ramp down portion of the MRI sequence. The slice select ramp down portion of the MRI sequence is defined as a period immediately subsequent to the RF pulse generation during which current in the at least one gradient coil is reduced to zero.

In accordance with an embodiment of the first aspect, the data for the navigator signal is captured after a slice select ramp down portion of the MRI sequence, when no current is passed through the at least one gradient coil, and before both a gradient refocus portion of the MRI sequence and a start of generation of image-encoding gradients for data acquisition.

In accordance with an embodiment of the first aspect, processing the navigator signal comprises filtering the navigator signal via an angular k-space filter.

In accordance with an embodiment of the first aspect, the filtering further comprises applying a temporal band-pass filter centered on a frequency corresponding to a common respiration rate.

In accordance with an embodiment of the first aspect, performing data acquisition using the MRI apparatus comprises gating image data according to a phase of the respiratory cycle identified using the processed navigator signal.

In accordance with an embodiment of the first aspect, the MRI sequence comprises a two dimensional (2D) or three-dimensional (3D) imaging sequence using a slice-selective excitation.

In accordance with an embodiment of the first aspect, the RF pulse comprises a slice-selective RF pulse. The slice-selective RF pulse can include a sinc or a ultra-short echo time (UTE) pulse.

In accordance with an embodiment of the first aspect, the MRI apparatus includes a plurality of RF receivers coupled to a plurality of RF coils. In addition, capturing the data for the navigator signal further comprises selecting an optimal RF coil from the plurality of coils to sample.

In accordance with a second aspect of the present disclosure, a magnetic resonance imaging (MRI) system is provided. The MRI system includes: an MRI apparatus; and a control system connected to the MRI apparatus. The control system includes at least one processor and a memory storing a set of instructions that, in response to being executed by the at least one processor, cause the MRI apparatus to: capture data for a navigator signal between a slice select portion of an MRI sequence and a gradient refocus portion of the MRI sequence, process the navigator signal to identify timing of a respiratory cycle of a subject being measured, and perform data reconstruction using the MRI apparatus based at least on the processed navigator signal. Performing data reconstruction can include performing data sorting and image formation based at least on the processed navigator signal. The slice select portion of the MRI sequence is a period where a current is passed through at least one gradient coil of the MRI apparatus while a radio frequency (RF) pulse is generated by a RF coil of the MRI apparatus

In accordance with an embodiment of the second aspect, the data for the navigator signal is captured during a slice select ramp down portion of the MRI sequence.

In accordance with an embodiment of the second aspect, wherein the data for the navigator signal is captured after a slice select ramp down portion of the MRI sequence, when no current is passed through at least one gradient coil, and before both a gradient refocus portion of the MRI sequence and a start of generation of image-encoding gradients for data acquisition.

In accordance with an embodiment of the second aspect, processing the navigator signal comprises filtering the navigator signal via an angular k-space filter.

In accordance with an embodiment of the second aspect, the filtering further comprises applying a temporal band-pass filter centered on a frequency corresponding to common respiration rates.

In accordance with an embodiment of the second aspect, performing data acquisition using the MRI apparatus comprises binning image data according to a phase of the respiratory cycle identified using the processed navigator signal.

In accordance with an embodiment of the second aspect, performing data acquisition using the MRI apparatus comprises gating image data according to a phase of the respiratory cycle identified using the processed navigator signal.

In accordance with an embodiment of the second aspect, the MRI sequence comprises a two-dimensional (2D) or three-dimensional (3D) imaging sequence using slice-selective excitation.

In accordance with an embodiment of the second aspect, the RF pulse comprises a slice-selective RF pulse.

In accordance with a third aspect of the present disclosure, a non-transitory computer-readable storage medium storing instructions is provided that, in response to being executed by one or more processors, cause a magnetic resonance imaging (MRI) apparatus to perform the method of the first aspect.

### BRIEF DESCRIPTION OF THE FIGURES

FIG. 1 illustrates a magnetic resonance imaging (MRI) system, in accordance with some embodiments of the present disclosure.

FIG. 2 illustrates a control system for the MRI system of FIG. 1, in accordance with some embodiments of the present disclosure.

FIG. 3A shows a timing diagram for an MRI sequence, in accordance with some embodiments of the present disclosure.

FIG. 3B shows the timing associated with three separate navigator signals, in accordance with some embodiments of the present disclosure.

FIG. 3C illustrates an MRI sequence corresponding with a 2D Cartesian trajectory, in accordance with some embodiments of the present disclosure.

FIG. 3D shows various signal processing steps applied to a captured raw navigator signal, in accordance with some embodiments of the present disclosure.

FIG. 3E illustrates the various navigator signals described herein, pre-and post-processing, in accordance with some embodiments of the present disclosure.

FIG. 4 shows a set of short-axis slice images from a 3D stack-of-spirals ultra-short echo-time acquisition, in accordance with some embodiments.

FIG. 5 illustrates a comparison between the four different navigator signals conducted during an experiment using ten subjects, in accordance with some embodiments.

FIG. 6 is a flowchart of a method for acquiring MRI images using navigator signals, in accordance with some embodiments of the present disclosure.

### DETAILED DESCRIPTION

Physiological information is used for MRI to guide acquisition timing for motion-resolved or motion-compensated imaging. Existing means for generating signals to track a patient's respiration cycle include use of devices on patients, such as electrodes for ECG (electrocardiogram) or pressure-sensitive respiratory bellows. Hardware modifications are also possible for radio frequency (RF) sensing, but this in not universally available.

Alternatively, modern advanced techniques for acquisition of free-breathing MRI images have two means to detect physiological motion in the data collection itself: 1) use of a navigation-only signal acquisition to determine the liver position, and 2) designing the image acquisition such that there is frequent sampling of a “direct-current” signal, at a timing where there is no spatial encoding and high MRI signal, that fluctuates with respiration. However, navigation segments can disrupt image acquisitions that rely on steady-state or fast imaging regimes, and implementing frequent sampling of “direct-current” signals can be difficult as well as not being possible to implement for every application.

The present disclosure describes acquisition of navigator signals by frequently sampling the signal emitted after the RF pulse and the slice select gradient refocus portion of the sequence, which has some unspecified spatial encoding-the “unintended” encoding present following a slice-selective pulse-used in the majority of MRI imaging techniques for choosing volumes of interest. As this unwanted signal is acquired during image acquisition “dead-time”, it is therefore an opportunity to acquire navigation information without disrupting the timing of the MRI sequence. In other words, while other existing techniques may require delay of the image acquisition during the MRI sequence, the technique described below can be implemented in parallel with image acquisition without disrupting the image data. It has been determined that there is physiological data present in the dead-time signal, and processing to use this signal for respiratory gating has been proven to be effective. As this signal can be acquired during the image data acquisition dead-time, this can be added to a number of MRI imaging sequences with minimal impact.

MRI applications used for determining respiratory motion, cardiac motion, and bulk subject motion, applied to cardiac imaging, pulmonary imaging, abdominal imaging, neuroimaging, musculoskeletal imaging are all within the scope of the present disclosure.

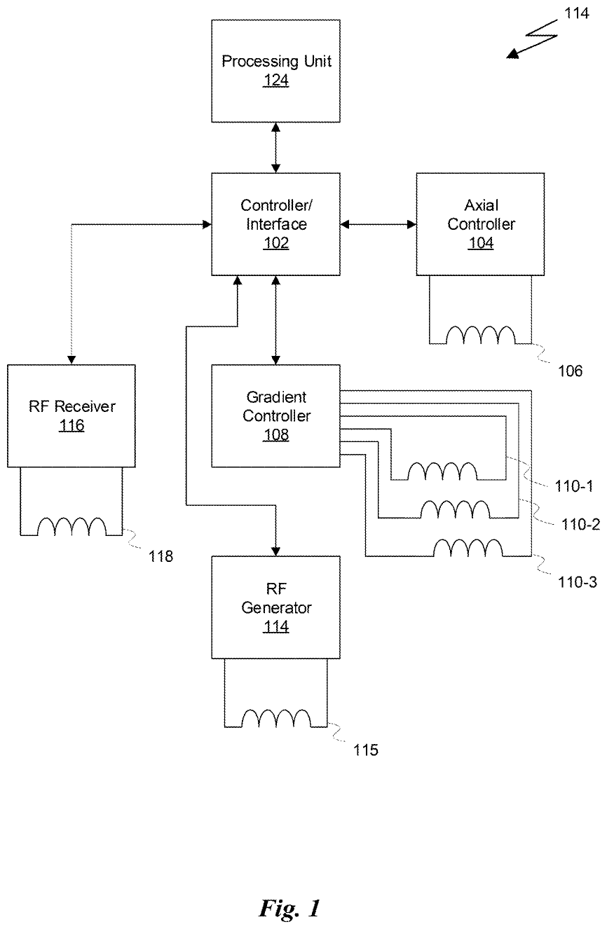

FIG. 1 illustrates an MRI system 100, in accordance with some embodiments of the present disclosure. Magnetic resonance (MR) measurements can be obtained using an MRI apparatus 100 as illustrated in FIG. 1. The MRI apparatus 100 includes a controller/interface 102 that is configured to apply selected magnetic fields such as constant or pulsed field gradients to an object (e.g., a patient) located proximate to the MRI apparatus 100. An axial controller 104 is in communication with an axial magnet 106 that is generally configured to produce a substantially constant magnetic field B₀ proximate the MRI apparatus 100. The axial magnet 106 may comprise a coil that includes superconducting material such as niobium-tin (Nb3Sn) or Magnesium Diboride (MgB2). A gradient controller 108 is configure to apply a constant or time-varying magnetic field gradient in a selected direction using a number of gradient coils 110. The gradient controller 108 may cause electrical current to be applied to the gradient coils 110 to apply diffusion or slice selection gradients to the object. The magnetic field gradient vector components may be referred to as Gₓ, G_{y}, and G_{z} corresponding to an x-axis, y-axis, and z-axis, respectively. In an embodiment, each axis is associated with a separate gradient coil, e.g., a first gradient coil 110-1 is associated with an x-axis, a second gradient coil 110-2 is associated with a y-axis, and a third gradient coil 110-3 is associated with a z-axis. A magnetic field gradient may be oriented in any direction, meaning the magnitude of the magnetic field changes along an axis corresponding to that direction, such that two or more magnetic field gradient vector components are non-zero.

An RF generator 114 is configured to generate one or more RF pulses of the electro-magnetic field to the object being imaged. The RF generator 114 is connected to one or more transmitter coils 115, and delivers an electrical current to the transmitter coils 115 to generate the RF pulse. One or more RF receivers 116 are coupled to corresponding receiver coils 118 and configured to detect or measure RF signals emitted by the object in response to the excitation from the RF pulse. Typically, the RF receiver 116 includes at least an amplifier and an analog-to-digital converter (ADC) to convert the received signal strength to a digital value to be processed by a computing device or other processing system.

The gradient controller 108 can be configured to produce pulses or other gradient fields, using the gradient coils 110, along one or more directions relative to the orientation of the MRI apparatus or the object. By selection of such gradients and via other applied RF pulses using the transmitter coils 115, various imaging or MRI sequences can be applied. An MRI sequence refers to a specific control scheme for generating pulses and or currents in the various coils to effectively acquire data to form at least one MRI image.

During imaging, the object or specimen may be divided into volume elements (voxels) and MR signals for a plurality of gradient directions are acquired. It will be appreciated that by selecting a magnetic field gradient along a specific direction and then exciting the components (e.g., atoms, molecules) of the object at a specific frequency of RF pulse, certain components of the object in a plane normal to the direction of the gradient may be excited more strongly than other components of the object further away from the plane. Thus, image data can be acquired by varying the gradient and RF pulses to measure a signal response corresponding to that slice and or location.

A processing device 124 such as a personal computer, a printed circuit board with one or more processors, memory, and/or other chipsets attached thereto, or the like, is provided for acquisition control and/or processing and analysis of collected data. The processing device 124 may include volatile (e.g., dynamic random access memory (DRAM)) or non-volatile (e.g., hard disk drive (HDD), solid state drive (SSD), flash memory, or the like) memory. The processing device 124 may also include a network interface controller (NIC) or other communications interface to communicate with other computing devices over, e.g., a network such as a local area network (LAN) or wide area network (WAN) such as the Internet. In some embodiments, data can be transmitted from the processing device 124 to a service (e.g., a service hosted on a cloud platform) for remote processing or storage. Signal acquisition, instrument control, and signal analysis can be performed locally on the processing device 124, or in a distributed computing environment using a plurality of processing devices connected via a network. For example, in some embodiments, signal acquisition and signal processing can be performed on different devices. The processing device 124 can also be configured to implement an interface (e.g., a graphical user interface (GUI)) that enables a technician or operator of the MRI apparatus to control the MRI sequence scheme to select magnetic field magnitudes, gradient field direction and magnitude, RF pulse frequency, duration, and/or shape, and the like. The processing device 124 can implement the interface and any other functions in computer-executable instructions capable of being executed by the one or more processors of the processing device 124, the computer-executable instructions stored in a memory, such as the HDD and loaded in the DRAM for execution. In addition, parameters for the various MRI sequence schema can be loaded into the memory as well. In general, control and data acquisition of the MRI apparatus 100 can be controlled locally or remotely via instructions and/or data transmitted remotely to the processing device 124 via a network connection.

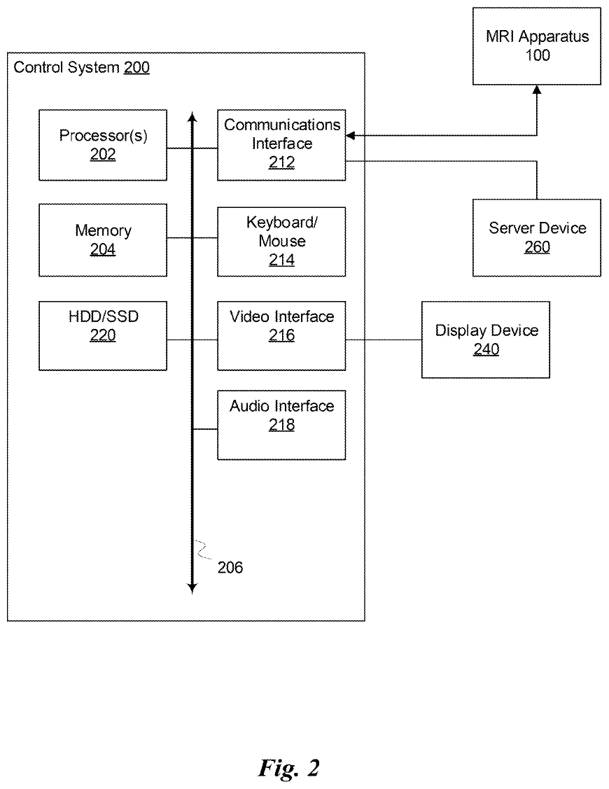

FIG. 2 illustrates a control system 200 for the MRI system 100 of FIG. 1, in accordance with some embodiments of the present disclosure. The control system 200 is used to implement the functions of the hardware described above for the MRI system 100. In some embodiments, implementation may be in the form of a set of computer-readable instructions executed by a computing device. However, any system that implements said functionality via any combination of hardware, firmware, or software is within the scope of the present disclosure. In addition, although the following description may be within the context of a single computing device, these functions can be implemented on a distributed system over multiple computing devices.

As shown in FIG. 2, the control system 200 includes one or more processors 202, a memory 204, and a communications bus 206. The communications bus 206 communicatively connects various system components, such as the processor(s) 202 and/or the memory 204. The memory 204 may include any combination of read only memory (ROM), random access memory (RAM), or non-volatile memory (e.g., HDD, SSD, Flash, etc.). The communications bus 206 may include any number of bus architecture types well-known in the art including but not limited to a peripheral component interconnect (PCI) bus, PCI Express (PCIe) bus, Universal Serial Bus (USB), or a dedicated chip-to-chip bus. The control system 200 may also include a number of input/output (I/O) devices and/or interfaces such as a communication interface 212 (e.g., network interface controller (NIC)), keyboard/mouse 214, a video interface 216 (e.g., HDMI, etc.), audio interface 218 (e.g., speakers, microphone, etc.), or the like.

The control system 200 may include software stored in non-volatile storage devices 220 (e.g., HDD, SSD, Flash, etc.), which may then be loaded into the memory 204. The software may include an operating system and one or more applications.

In some embodiments, the memory 204 may also store program modules and/or data that are loaded and/or used by at least one of the applications. The program modules may include software add-ons that add customized functionality to an application. The data may include files or other data structures storing information such as parameters that define an MRI sequence.

A GUI may be displayed on a display device 240. The GUI may allow a technician or operator to enter commands or values to control the MRI apparatus 100, start or stop an MRI image acquisition sequence, view images generated in response to measured signals, and the like. Commands or values can be entered manually via the one or more I/O devices. In some embodiments, the display device 240 may include a touch-sensitive screen that allows a technician or operator to enter commands directly using the display device 240.

Again, the control system 200 may include a communications interface 212 to enable the control system 200 to communicate with other remote devices, such as the MRI apparatus 100 or a server device 260, over a network. The network interface may be wired (e.g., IEE 802.3 Ethernet) or wireless (e.g., 802.11 WiFi). In some embodiments, the network interface may be an optical interface (e.g., SONET). In other embodiments, the control system 200 may be directly connected to the MRI apparatus 100 via a wired interface.

### MRI Image Acquisition Using Navigator Signals

When capturing images using the MRI apparatus 100, it may be necessary to keep the patient or subject being imaged relatively still in order to get better quality images. However, while a person breathes, there will necessarily be motion of the subject's body and internal organs as the diaphragm moves and the lungs inhale or exhale air. Traditionally, images with reduced motion artifacts were acquired by requesting the patient to hold their breath while the image data was acquired. Newer techniques use navigator signals that detect motion of the patient due to respiration and time image acquisition or inform image post-processing accordingly, allowing the subject to continue breathing normally while image data is acquired. Timing the acquisition of image data to match up with a particular phase of the subject's respiratory cycle can help to improve image quality, although this can also extend the time required to capture all of the necessary image data. A novel method for capturing the state of the person's respiratory cycle during image acquisition while not unduly extending the total period required to acquire all of the necessary image data can help ease the comfort of the subject and save time to allow for more subjects to be imaged over the course of a day.

Conventionally, a navigator signal has been captured using, e.g., an ECG based on electrodes placed on the subject's body or using bellows that capture the chest motion during inspiration and expiration. However, these techniques are not as comfortable to the patient and take additional steps to set up by a technician. Another technique for generating a navigator signal uses the MRI system itself to capture images of a particular structure in the body (e.g., the person's diaphragm, liver, etc.) in order to capture the person's respiratory cycle due to motion of these structures. This typically involves capturing at least one image at a slice position corresponding to these structures and then tracking motion by analyzing a region of interest in the image. However, these navigator signals were captured during normal image acquisition phase of the MRI sequence and may have added significant delays to capturing of the intended images.

Free-breathing image acquisition is an ideal solution for robustness and patient comfort. A DC signal can be sampled each repetition time (TR) as a trajectory crosses k-space center, and this signal can be used as a free-breathing navigator signal to determine physiological motion. However, with 2D or 3D “stack” trajectories, the DC signal is measured less frequently, and may not have sufficient sampling frequency to resolve the respiratory cycle. As such, traditional trajectories (especially 3D Cartesian, 3D stack-of-spirals, and 3D stack-of-stars trajectories) may need to be modified, thereby extending the required image acquisition time, in order to use a DC navigator signal. In contrast, as proposed herein, a potentially “free” self-navigator signal can be captured by measuring the signal immediately following an RF pulse for imaging acquisition. This navigator signal can be implemented with minimal or no penalty in acquisition efficiency. In an example application, the navigator signal can be used for respiratory binning of a 3D stack-of-spiral image acquisition trajectory.

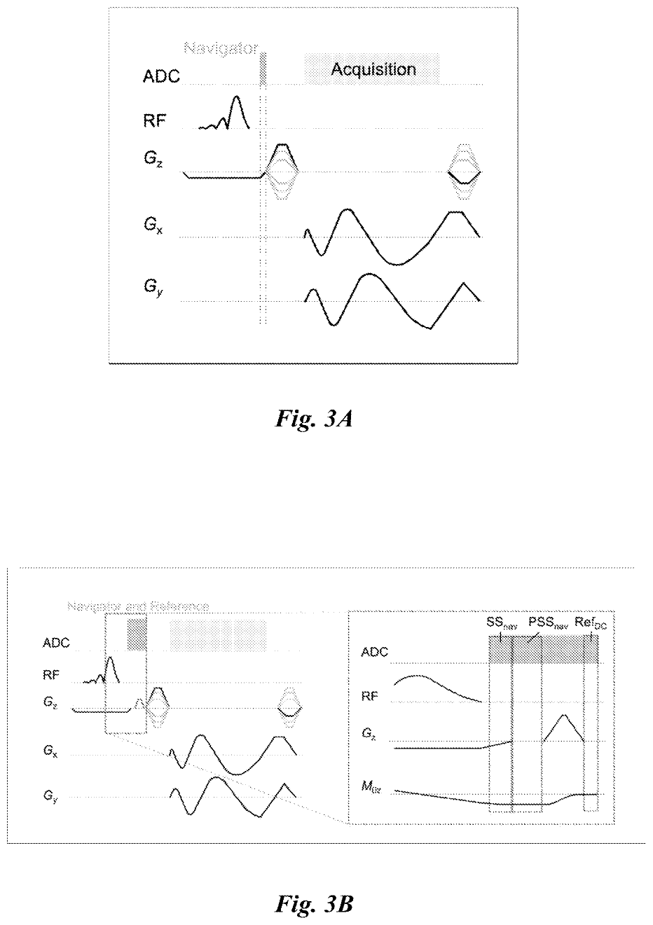

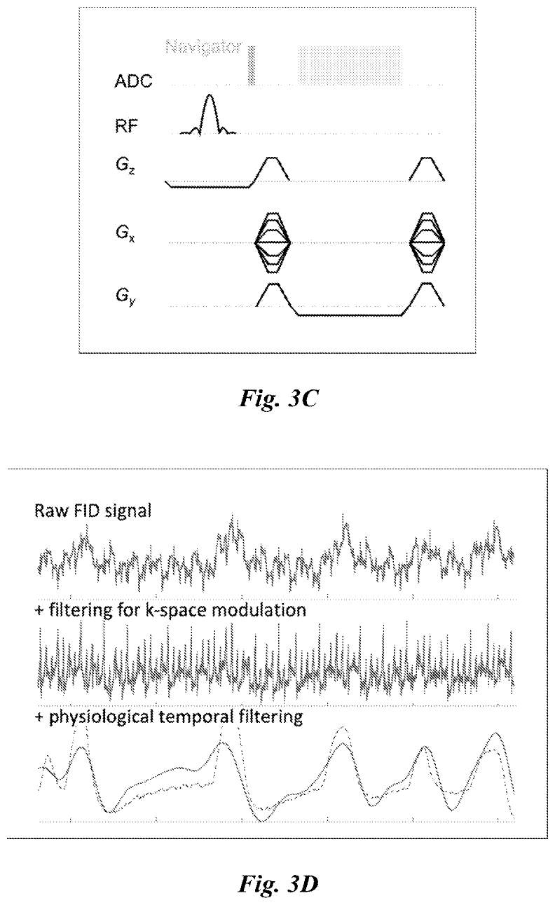

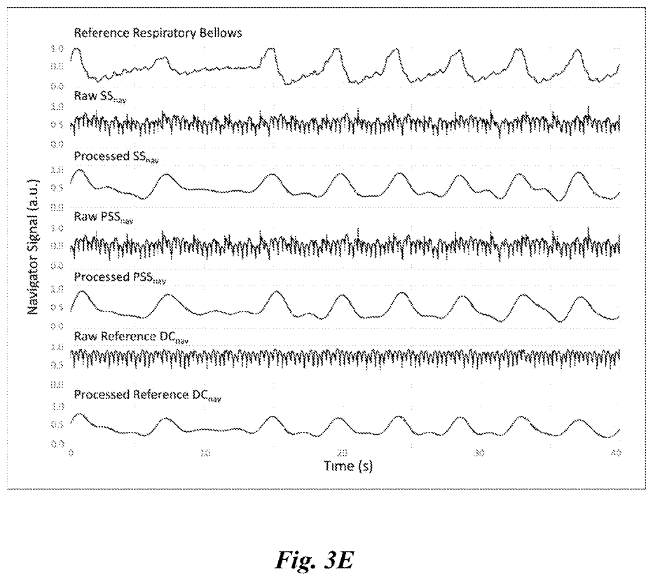

FIG. 3A shows a timing diagram for an MRI sequence, in accordance with some embodiments of the present disclosure. The MRI sequence is implemented by generating a number of signals in a particular order in time. The signals shown in FIG. 3A include an ADC signal that indicates when the receiver coil 118 is being sampled by the ADC in the RF receiver 116; an RF signal that indicates when the RF pulse is generated by the RF generator 114; and Gradient signals (e.g., Gₓ, G_{y}, G_{z}) that indicate the magnetic field gradients generated by the gradient controller 108 and the gradient coils 110. The MRI sequence shown in FIG. 3A corresponds to a 3D stack-of-spirals acquisition trajectory within which the navigator signal (SSₙₐᵥ) is sampled during the slice-select ramp-down portion of the MRI sequence and before the image-encoding gradients begin. Image encoding refers to the portion of the MRI sequence where spatial information is encoded by varying the current in one or more gradient coils to implement the k-space trajectory for image acquisition. This follows the slice select and/or slice rewinder portion of the MRI sequence.

The MRI sequence begins with slice select by generating a gradient signal in the G_{z} coil 110-3. Although the gradient shown here is axial along the z-axis, the gradient may be generated in any particular orientation using one or more of the gradient coils 110. Once the magnetic field gradient has been set, the RF pulse is generated in the RF generator 114. After the RF pulse is complete, slice select ramp down begins by ramping down the current in the G_{z} coil 110-3. It is during this stage of the MRI sequence that a “free” slice-select navigator (SSₙₐᵥ) signal can be sampled.

FIG. 3B shows the timing associated with three separate navigator signals in accordance with some embodiments of the present disclosure. The navigator signals are referred to as: SSₙₐᵥ; PSSₙₐᵥ, and a MRI reference DCₙₐᵥ. The SSₙₐᵥ signal is generated by sampling the ADC signal during a slice select ramp down portion of the MRI sequence before the current in the G_{z} coil is reduced to zero. The PSSₙₐᵥ signal is generated while no current is passing through the G_{z} coil to reduce any signal variations due to the changing magnetic field gradient. The SSₙₐᵥand PSSₙₐᵥ signals may be referred to herein as “free” navigator signals because they can be collected in a time in the MRI sequence that requires no or minimal delay to the normal image acquisition timing of most traditional MRI sequences. The slice-select zeroth gradient moment M_{0z} (the gradient time integral) demonstrated in the detailed view in FIG. 3B shows different amounts of spatial encoding between the different navigator signals. The DCₙₐᵥ signal is generated after the current in the G_{z} coil is briefly reversed to refocus the zeroth gradient moment at M_{0z}=0. While the DCₙₐᵥ signal is shown here for comparison to the “free” navigator signals, it may not be an ideal navigator signal because some MRI sequences do not refocus the magnetic field at sufficient frequency to be able to capture enough data to use as a navigator signal.

It will be appreciated that the proposed navigator signals, SSₙₐᵥ and PSSₙₐᵥ are collected before the image-encoding gradients are applied during normal MRI acquisition. There is no delay or minimal delay to image acquisition using the “free” navigator signals unlike significant delays added in other common methods for collecting navigator signals such as those that rely on image processing to identify the liver location or motion or an intermittent superior-inferior signal acquisition. In some MRI sequences, sometimes referred to as speed-optimal imaging sequences using Cartesian trajectories, where image-encoding may begin during slice select ramp down, there may be a minimal penalty incurred due to delay of starting the image-encoding gradients until the navigator signal has been sampled.

It will be appreciated that the MRI sequence shown in FIG. 3B has been purposefully modified to require slice rewinder during each repetition time TR in order to generate a period for sampling the reference DCₙₐᵥ signal. In practice, many MRI sequences do not include this slice rewinder each TR, and so this navigator signal may not be captured without modifying the current MRI sequences.

FIG. 3C illustrates an MRI sequence corresponding with a 2D Cartesian trajectory, in accordance with some embodiments of the present disclosure. The SSₙₐᵥ signal is demonstrated within the timing of the 2D Cartesian MRI sequence acquisition. The SSₙₐᵥ signal is placed during the slice-select ramp down portion of the MRI sequence, which requires that the image-encoding gradients (Gₓ and G_{y}) be delayed until the start of the slice-select rewinding gradient after the navigator signal is acquired.

FIG. 3D shows various signal processing steps applied to a captured raw navigator signal, in accordance with some embodiments of the present disclosure. As shown in FIG. 3D, the raw navigator signal can be filtered by an angular filter for k-space modulation, such as described in Di Sopra et al., “An automated approach to fully self-gated free-running cardiac and respiratory motion-resolved 5D whole-heart MRI,” J. Magnetic Resonance in Medicine, Vol. 82:6 (December 2019), which is herein incorporated by reference in its entirety. In addition, temporal filtering can also be applied to remove any non-respiratory based temporal variations. For example, a band-pass filter can be applied to filter out variations in the signal that don't appear to be cyclic at the same rate of a person's respiration cycle. Alternatively, any frequency response or temporal filter band may be selected that is appropriate for a given subject for common respiratory rates.

FIG. 3E illustrates the various navigator signals described herein, pre-and post-processing, in accordance with some embodiments of the present disclosure. As shown in FIG. 3E, the collected “free” navigator signals are compared to a conventional navigator signal generated based on a bellows placed on a patient's chest. The DCₙₐᵥ signal is also shown for reference. It can be seen that the “free” navigator signals appear to capture the patient's respiratory cycle with a similar effectiveness as compared to the traditional bellows navigator signal and/or the DCₙₐᵥ signal.

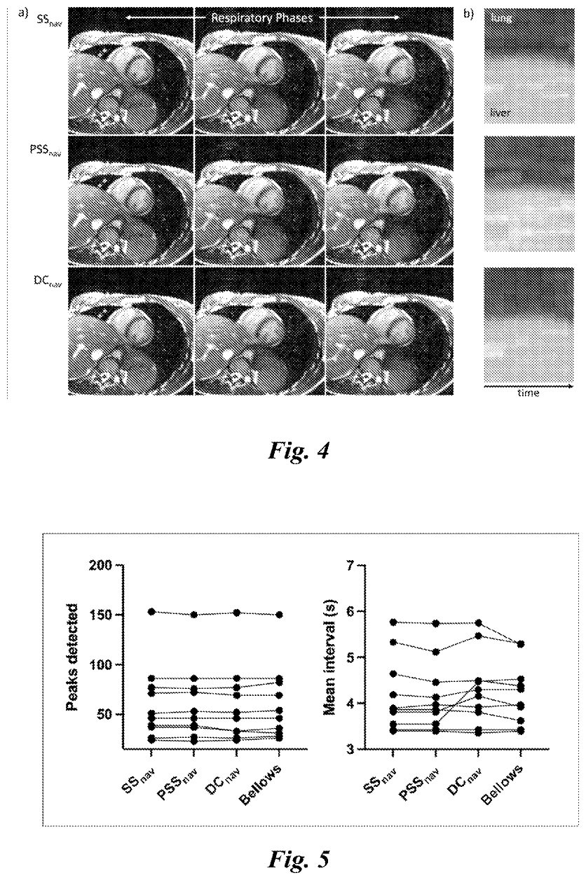

FIG. 4 shows a set of short-axis slice images from a 3D stack-of-spirals ultra-short echo-time (UTE) acquisition, in accordance with some embodiments. It will be appreciated that the navigator signals described above (e.g., SSₙₐᵥ or PSSₙₐᵥ) may be incorporated into a number of common MRI sequences. In particular, these navigator signals may be particularly suited for capturing a 2D trajectory or 3D stack trajectories associated with a slice selective pulse such as a sinc pulse or UTE pulse. Such MRI sequences are not particularly suited for conventional DC-based navigator signals as the trajectories in those pulse sequences do not return to the center of k-space at sufficient frequency to capture the respiratory cycle. In contrast, the SSₙₐᵥ and PSSₙₐᵥ signals can be captured without first returning to the center of k-space and, therefore, are particularly suited to 2D or 3D stack trajectories. As shown in FIG. 4, the slice images have been resolved for respiratory phase (highlighted by temporal line profiles depicting the lung-liver interfact in FIG. 4b, drawn from the dashed line in FIG. 4a), as determined by the SSₙₐᵥ, PSSₙₐᵥ and reference DCₙₐᵥ signal acquired every repetition time.

FIG. 5 illustrates a comparison between the four different navigator signals conducted during an experiment using ten subjects, in accordance with some embodiments. The charts shown in FIG. 5 indicate the number of peaks detected (i.e., respiratory cycles) and a mean interval between peaks (i.e., average duration of the respiratory cycle), in seconds, over a 3 minute image acquisition interval for the four different subjects. When comparing the different navigator signals, no significant difference was found between the number of peaks detected (p>0.43, ANOVA), and no significant differences were found between mean respiratory interval (p>0.31, ANOVA).

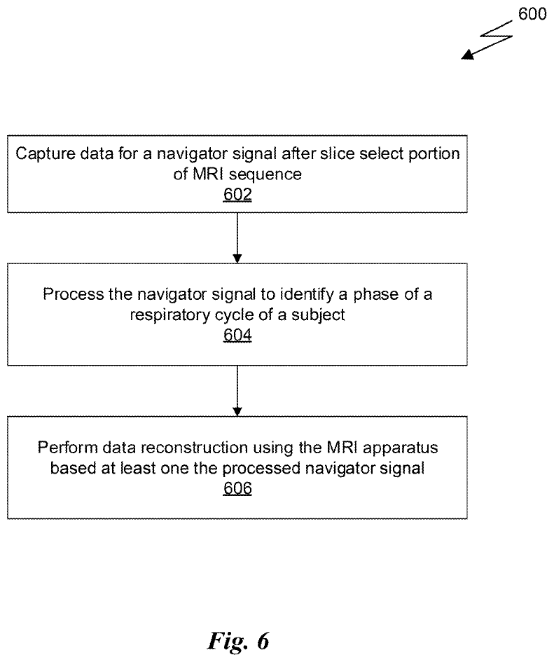

FIG. 6 is a flowchart of a method 600 for acquiring MRI images using navigator signals, in accordance with some embodiments of the present disclosure. The method 600 can be implemented one or more processors of the control system 200. However, those of skill in the art will recognize that any system suitable of controlling an MRI apparatus is contemplated as being within the scope of the present disclosure. In an embodiment, the method 600 can be implemented as a set of instructions that are executed by one or more processors of a computing device that is communicatively connected to an MRI apparatus.

At 602, data for a navigator signal is captured using an MRI apparatus. In an embodiment, the navigator signal is captured by sampling (e.g., using an ADC) an RF receiver 116 connected to an RF coil 118. The sample can include a number of data points captured every TR (sampling cycle) to capture MRI signal that includes some undesigned slice-encoding post slice-excitation, which can reduce the signal amplitude. As this signal will be proportional to the image volume being captured, the navigator signal over time will include motion within the imaging volume. The frequent sampling every TR drives the filtering for the low signal navigator such that the physiological motion can be extracted from the filtering process.

In an embodiment, the MRI apparatus may include multiple RF receiver coils 118 (which may each be coupled to a separate RF receiver 116 circuit) disposed at different locations proximate to the subject being imaged. In such cases, capturing the data for the navigator signal may further comprise selecting an optimal RF coil from the plurality of coils 118 to sample. Selecting the optimal RF coil can include comparing a power output of the signal received by each RF coil and selecting the RF coil corresponding with the highest power output as the optimal coil or selecting the coil that exhibits the most dominant motion within the imaging volume, such as described in Zhang et al., “Robust Self-Navigated Body MRI using Dense Coil Arrays,” J. Magnetic Resonance in Medicine, Vol. 76:1 (July 2016), which is incorporated by reference herein in its entirety.

In an embodiment, the navigator signal is captured at a time in the MRI sequence between a slice select portion of the MRI sequence and a gradient refocus portion of the MRI sequence. The slice select portion of the MRI sequence is a period where a current is passed through at least one gradient coil of the MRI apparatus while a radio frequency (RF) pulse is generated by a RF coil of the MRI apparatus. Once the RF pulse is complete, and as the current in the gradient coil is ramping down to zero current, a SSₙₐᵥ signal can be captured by the MRI apparatus. Alternatively, in another embodiment, the navigator signal can be captured after slice select ramp down is complete (i.e., after the current in the gradient coil reaches zero amps) but prior to a gradient refocus portion of the MRI sequence. This period of the MRI sequence can be referred to as a free induction decay period, and the navigator captured at this time is referred to as the PSSₙₐᵥ signal. The gradient refocus portion refers to a brief period after slice select encoding where the current in the gradient coil is reversed to return the magnetic field to a DC state.

At 604, the navigator signal is processed to identify timing of a respiratory cycle of a subject being measured. In an embodiment, the navigator signal can be filtered via an angular k-space filter. In some embodiments, the navigator signal can also be filtered by a temporal band-pass filter. Alternatively, other types of filters can be applied, such as a low-pass or high-pass temporal filter.

At 606, data reconstruction is performed using the MRI apparatus based at least one the processed navigator signal. Image signals are collected in a conventional manner after the navigator signal has been sampled. In an embodiment, the navigator signal can be utilized retroactively to generate MRI images for display or storage. The navigator signal can be used to identify a phase of a respiratory cycle corresponding to the image data captured during each repetition time (TR). In one case, the image data for a plurality of RF pulses over a plurality of TR periods can be binned according to the different phases of the respiratory cycle. The image data for a particular bin (i.e., corresponding to a particular phase of the respiratory cycle) is then used to reconstruct an image or images corresponding to that particular phase of the respiratory cycle. As such, a plurality of images are generated corresponding to a plurality of different phases of the respiratory cycle, thereby increasing the chances that motion artifacts are subdued or resolved.

It will be appreciated that the steps of the method 600 maybe repeated for each TR of an MRI sequence in order to capture data points for the desired k-space trajectories. The navigator signals may be used with a number of different types of trajectories, including, e.g., Cartesian, radial and spiral trajectories. In particular, the navigator-based techniques described above are particularly suited for use with two-dimensional (2D) or three-dimensional (3D) imaging sequence using a stack trajectories in k-space. Furthermore, the navigator-based technique may also be suitable for use with slice-selective RF pulse types such as sinc or ultrashort echo time (UTE).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method applied using a magnetic resonance imaging (MRI) apparatus, the method comprising:
capturing data for a navigator signal between a slice select portion of an MRI sequence and a gradient refocus portion of the MRI sequence, wherein the slice select portion of the MRI sequence is a period where a current is passed through at least one gradient coil of the MRI apparatus while a radio frequency (RF) pulse is generated by a RF coil of the MRI apparatus;
processing the navigator signal to identify timing of a respiratory cycle of a subject being measured; and
performing data reconstruction using the MRI apparatus based at least on the processed navigator signal.

2. The method of claim 1, wherein the data for the navigator signal is captured during a slice select ramp down portion of the MRI sequence.

3. The method of claim 1, wherein the data for the navigator signal is captured after a slice select ramp down portion of the MRI sequence, when no current is passed through the at least one gradient coil, and before both a gradient refocus portion of the MRI sequence and a start of generation of image-encoding gradients for data acquisition.

4. The method of claim 1, wherein processing the navigator signal comprises filtering the navigator signal via an angular k-space filter.

5. The method of claim 4, wherein the filtering further comprises applying a temporal band-pass filter centered on a frequency corresponding to a common respiration rate.

6. The method of claim 4, wherein the MRI apparatus includes a plurality of RF receivers coupled to a plurality of RF coils, and wherein capturing the data for the navigator signal further comprises selecting an optimal RF coil from the plurality of coils to sample.

7. The method of claim 1, wherein performing data acquisition using the MRI apparatus comprises binning image data according to a phase of the respiratory cycle identified using the processed navigator signal.

8. The method of claim 1, wherein the MRI sequence comprises a two-dimensional (2D) or three-dimensional (3D) imaging sequence using a stack trajectory in k-space.

9. The method of claim 1, wherein the RF pulse comprises a slice-selective pulse.

10. A magnetic resonance imaging (MRI) system, the MRI system comprising:
an MRI apparatus; and
a control system connected to the MRI apparatus, wherein the control system includes at least one processor and a memory storing a set of instructions that, in response to being executed by the at least one processor, cause the MRI apparatus to:
capture data for a navigator signal between a slice select portion of an MRI sequence and a gradient refocus portion of the MRI sequence, wherein the slice select portion of the MRI sequence is a period where a current is passed through at least one gradient coil of the MRI apparatus while a radio frequency (RF) pulse is generated by a RF coil of the MRI apparatus;
process the navigator signal to identify timing of a respiratory cycle of a subject being measured; and
perform data reconstruction using the MRI apparatus based at least on the processed navigator signal.

11. The MRI system of claim 10, wherein the data for the navigator signal is captured during a slice select ramp down portion of the MRI sequence.

12. The MRI system of claim 10, wherein the data for the navigator signal is captured after a slice select ramp down portion of the MRI sequence, when no current is passed through the at least one gradient coil, and before both a gradient refocus portion of the MRI sequence and a start of image-encoding gradients for data acquisition.

13. The MRI system of claim 10, wherein processing the navigator signal comprises filtering the navigator signal via an angular k-space filter.

14. The MRI system of claim 13, wherein the filtering further comprises applying a temporal band-pass filter centered on a frequency corresponding to a common respiration rate.

15. The MRI system of claim 10, wherein performing data reconstruction using the MRI apparatus comprises binning image data according to a phase of the respiratory cycle identified using the processed navigator signal.

16. The MRI system of claim 10, wherein the MRI sequence comprises a two-dimensional (2D) or three-dimensional (3D) imaging sequence using a stack trajectory in k-space.

17. The MRI system of claim 10, wherein the RF pulse comprises of a slice-selective pulse.

18. A non-transitory computer-readable storage medium storing instructions that, in response to being executed by one or more processors, cause a magnetic resonance imaging (MRI) apparatus to:
capture data for a navigator signal between a slice select portion of an MRI sequence and a gradient refocus portion of the MRI sequence, wherein the slice select portion of the MRI sequence is a period where a current is passed through at least one gradient coil of the MRI apparatus while a radio frequency (RF) pulse is generated by a RF coil of the MRI apparatus;
process the navigator signal to identify timing of a respiratory cycle of a subject being measured; and
perform data reconstruction using the MRI apparatus based at least on the processed navigator signal.

19. The non-transitory computer-readable storage medium of claim 18, wherein the data for the navigator signal is captured during a slice select ramp down portion of the MRI sequence.

20. The non-transitory computer-readable storage medium of claim 18, wherein the data for the navigator signal is captured after a slice select ramp down portion of the MRI sequence, when no current is passed through the at least one gradient coil, and before both a gradient refocus portion of the MRI sequence and a start of generation of image-encoding gradients for data acquisition.
